(19) United States
(12) Patent Application Publication
(10) Pub. No.: US 2021/0290522 A1
(43) Pub. Date: Sep. 23, 2021

(54) MONO-DOSE PRODUCTS, SYSTEMS, AND METHODS FOR KERATINOUS SUBSTRATES

(71) Applicant: L'Oreal, Paris (FR)

(72) Inventors: Aditi GOGINENI, Hoboken, NJ (US); Aline GUIMONT, South Orange, NJ (US); Brian Anthony MEDEIROS, Windham, NJ (US)

(21) Appl. No.: 17/204,411

(22) Filed: Mar. 17, 2021

Related U.S. Application Data
(60) Provisional application No. 62/991,547, filed on Mar. 18, 2020.

(30) Foreign Application Priority Data
Apr. 28, 2020 (FR) 2004204

(51) Int. Cl. A61K 8/92; A61Q 5/02; A61Q 19/10; A61K 8/73; A61K 8/46; A61K 8/41; A61K 8/34

(52) U.S. Cl. CPC A61K 8/922; A61Q 5/02; A61Q 19/10; A61K 8/927; A61K 8/737; A61K 2800/42; A61K 8/466; A61K 8/463; A61K 8/416; A61K 8/342; A61K 2800/5426; A61K 8/731

(57) ABSTRACT

Disclosed are mono-dose products and systems for caring for, cleansing, and/or conditioning keratinous substrates, comprising an internal core formulation comprising at least one component chosen from surfactants and conditioning agents, a protective shell coating comprising at least one structuring agent, and optionally a water-resistant coating comprising at least one wax. Also disclosed are methods of caring for, cleansing, and/or conditioning the skin, hair, and/or scalp with the mono-dose products and systems.

### TECHNICAL FIELD

The disclosure relates to products, systems, and methods for caring for, cleansing, and/or conditioning keratinous substrates, such as skin, hair, and/or scalp.

### BACKGROUND

Individuals desire healthy skin and hair, as healthy-looking skin and hair is considered to be a sign of good health and good hygiene. Thus, caring for, cleansing, and/or conditioning the skin and hair is routine. At the same time, consumers also desire to reduce their individual environmental footprint, which can include, for example, seeking products having reduced amounts of packaging waste and/or products which allow for reduced CO₂ emissions associated therewith.

In order to address these concerns, the present disclosure relates to mono-dose products and systems that are useful in caring for, cleansing, and/or conditioning the skin, hair, and/or scalp, which may contribute to a reduced environmental footprint. The mono-dose products and systems may have one or more of the following advantages: reduced waste (e.g. uses less or no plastic), reduced CO₂ emissions associated with transportation of the product (e.g. due to reduced weight), ease of transport or packing during travel, ease of consumer use and/or ease of distribution of product onto the skin, hair, and/or scalp, increased hygiene due to single-use nature, reduced cost, and/or humidity resistance which allows the product to be stored in the bathroom or shower.

### SUMMARY

According to various embodiments, the disclosure relates to mono-dose products and systems for caring for, cleansing, and/or conditioning keratinous substrates, the mono-dose systems comprising one or more mono-dose products having (a) an internal core formulation comprising at least one component chosen from surfactants and conditioning agents, (b) a protective shell coating comprising at least one structuring agent, and optionally (c) a water-resistant coating.

In further embodiments, the disclosure relates to methods of caring for, cleansing, and/or conditioning the skin, hair, and/or scalp with the mono-dose products and systems.

### BRIEF DESCRIPTION OF FIGURE

FIG. 1 shows a three-part mono-dose product having an internal core formulation (100), a protective shell coating (200), and a water-resistant coating (300), according to an exemplary embodiment of the disclosure.

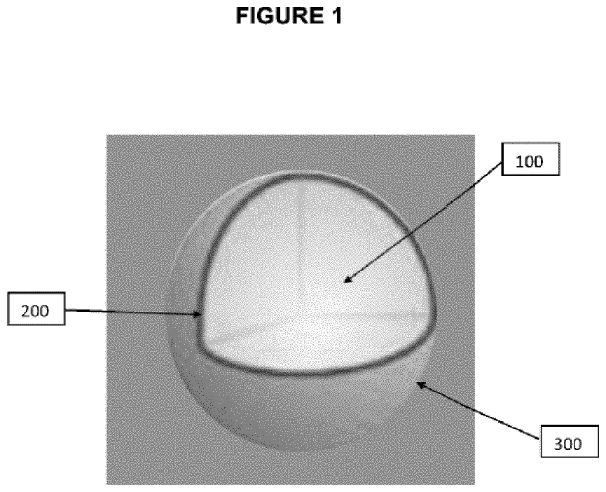

### DESCRIPTION

The disclosure relates, in various embodiments, to mono-dose products and systems for caring for, cleansing, and/or conditioning keratinous substrates such as the skin, hair, and/or scalp. The mono-dose systems comprise one or more mono-dose products. The mono-dose products may optionally be two-part products, three-part products, or more. The mono-dose products comprise: (a) a first part comprising an internal core care, cleansing, and/or conditioning formulation; (b) a second part comprising a protective shell coating; and optionally (c) a third part comprising a water-resistant coating. The disclosure also relates to methods of caring for, cleansing, and/or conditioning the skin, hair, and/or scalp using the mono-dose products and systems.

### Internal Core Formulation

The internal core formulation may be a formulation for caring for, cleansing, and/or conditioning keratinous substrates such as skin, hair, and/or scalp, and may comprise surfactants and/or conditioning agents. The internal core formulation may, in various exemplary and non-limiting embodiments, be a “shampoo” formulation, a “conditioning shampoo” formulation, an “all-in-one conditioning and shampooing” formulation, a “cream shampoo” formulation, a “cream rinse” formulation, a “cleansing cream” formulation, a “body cleanser (or body wash)” formulation, a “skin cleanser” formulation, a “facial cleanser” formulation, an “all-in-one skin or face conditioning and cleansing” formulation, a “cleansing scrub” formulation, a “skin moisturizer” formulation, or the like.

For example, in various embodiments, the mono-dose products and systems may be primarily skin and/or hair cleansing systems, and therefore the internal core formulation may comprise mainly surfactants. In further embodiments, the mono-dose products and systems may be skin and/or hair cleansing and conditioning systems, and therefore the internal core formulation may comprise both surfactants and conditioning agents. In yet further embodiments, the mono-dose products and systems may be primarily hair conditioning systems, and therefore the internal core formulation may comprise mainly conditioning agents. In still further embodiments, the mono-dose products and systems may be primarily skin cleansing systems, and therefore the internal core formulation may comprise mainly surfactants. In further embodiments still, the mono-dose products and systems may be systems for moisturizing the skin, and therefore the internal core formulation may comprise mainly conditioning (moisturizing) agents. A person skilled in the art will be able to determine the components and amounts thereof for each mono-dose product, based on the desired use and/or results, and can adjust the internal core formulation accordingly.

The internal core formulation may, in certain embodiments, be a semi-soft, concentrated formulation, e.g. may be gel-like, or semi-solid, or may have the consistency of a thick cream, thick emulsion, or gel-cream. By “gel-like” it is meant that the compositions may have increased viscosity relative to conventional compositions for caring for, cleansing, and/or conditioning keratinous substrates, or may have a gum-like or jello-like consistency. In some embodiments, the internal core formulation may have semi-solid viscoelastic properties and/or structure.

### Surfactants

The internal core formulations may optionally comprise at least one surfactant. Surfactants are widely used in personal care cleansing products (e.g., shampoos, body washes, facial cleansers, liquid hand soaps, etc.) because of the cleansing attributes provided. The at least one surfactant may be chosen from anionic surfactants, cationic surfactants, amphoteric or zwitterionic surfactants, and non-ionic surfactants.

In various embodiments, the at least one surfactant may be chosen from anionic surfactants. By “anionic surfactants” it is meant a surfactant comprising, as ionic or ionizable groups, anionic groups. These anionic groups may, for example, be chosen from CO2H, CO2⁻, SO3H, SO3⁻, OSO3H, OSO3⁻, H2PO3⁻HPO3⁻, PO3²⁻, H2PO2⁻, HPO2⁻, PO2²⁻, POH and PO⁻ groups.

As nonlimiting examples of anionic surfactants that may be used in the internal core formulation, mention may be made of alkyl sulfates, alkyl ether sulfates, alkylamido ether sulfates, alkylarylpolyether sulfates, monoglyceride sulfates, alkylsulfonates, alkylamidesulfonates, alkylarylsulfonates, α-olefin sulfonates, paraffin sulfonates, alkylsulfosuccinates, alkylether sulfosuccinates, alkylamide sulfosuccinates, alkylsulfoacetates, acylsarcosinates, acylglutamates, alkylsulfosuccinamates, acylisethionates and N—(C1-C4)alkyl N-acyltaurates, salts of alkyl monoesters of polyglycoside-polycarboxylic acids, acyllactylates, D-galactoside uronic acid salts, alkyl ether carboxylic acid salts, alkylaryl ether carboxylic acid salts, alkylamido ether carboxylic acid salts; and the corresponding non-salified forms of all these compounds; the alkyl and acyl groups of all these compounds (unless otherwise mentioned) comprising from 6 to 24 carbon atoms and the aryl group denoting a phenyl group. These compounds may, in some embodiments, be oxyethylenated, for example comprising from 1 to 50 ethylene oxide units.

By way of example only, the at least one anionic surfactant may be chosen from isethionate surfactants. According to various embodiments, the at least one isethionate surfactant may be chosen from acyl isethionates of the following formulae (I) or (II):

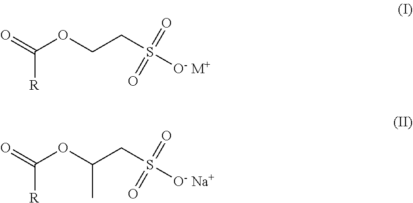

wherein R is chosen from H or an alkyl chain having from 1 to 30 carbon atoms, such as 6 to 24 carbon atoms, for example 8 to 20 carbon atoms, said chain being saturated or unsaturated, linear or branched, and M⁺ is a cation. Although sodium is shown as the cation in formula (II), it should be understood that the cation for both formula (I) and formula (II) may be any alkali metal ion such as sodium or potassium, ammonium ions, or alkanolammonium ions such as monoethanolammonium or triethanolammonium ions.

By way of non-limiting example, suitable acyl isethionate surfactants may include the reaction product of fatty acids esterified with isethionic acid and neutralized with sodium hydroxide. For example, acyl isethionates surfactants may be prepared by the reaction of an isethionate salt such as metal or ammonium isethionate and an a saturated or unsaturated, straight or branched, alkyl or alkenyl chain fatty acid having from 6 to 30 carbon atoms, preferably from 8 to 22 carbon atoms, more preferably from 6 to 18 carbon atoms. Optionally, a mixture of aliphatic fatty acids may be used for the preparation of commercial fatty acyl isethionates surfactants. Suitable fatty acids for isethionate surfactants can be derived from coconut oil or palm kernel oil, for instance.

Non-limiting examples of acyl isethionate surfactants that may be used include sodium lauroyl isethionate, sodium lauroyl methyl isethionate, sodium oleoyl isethionate, sodium oleoyl methyl isethionate, sodium stearoyl isethionate, sodium stearoyl methyl isethionate, sodium myristoyl isethionate, sodium myristoyl methyl isethionate, sodium palmitoyl isethionate, sodium palmitoyl methyl isethionate, sodium cocoyl isethionate, sodium cocoyl methyl isethionate, a blend of stearic acid and sodium cocoyl isethionate, ammonium cocoyl isethionate, ammonium cocoyl methyl isethionate, and mixtures thereof.

As further non-limiting examples, sulfate-based anionic surfactants may be chosen from, for example, alkyl sulfates, alkyl ether sulfates, alkylamido ether sulfates, alkylaryl polyether sulfates, monoglyceride sulfates, or mixtures thereof. In various embodiments, the anionic surfactant may be chosen from (C₆-C₂₄)alkyl sulfates, (C₆-C₂₄)alkyl ether sulfates, which are optionally ethoxylated, comprising from 2 to 50 ethylene oxide units, and mixtures thereof, in particular in the form of alkali metal salts or alkaline-earth metal salts, ammonium salts or amino alcohol salts. More preferentially, the anionic surfactant(s) are chosen from (C₁₀-C₂₀)alkyl ether sulfates, and in particular sodium lauryl ether sulfate, optionally containing 2.2 mol of ethylene oxide. In other embodiments, sodium lauryl sulfate, sodium lauryl ether sulfate, ammonium lauryl sulfate, ammonium lauryl ether sulfate, sodium laureth sulfate, or mixtures thereof may be chosen.

In various exemplary embodiments, the anionic surfactant may be chosen from non-sulfate anionic surfactants, such as, for example, alkylsulfonates, alkylamide sulfonates, alkylarylsulfonates, alpha-olefin sulfonates, paraffin sulfonates, alkylsulfosuccinates, alkyl ether sulfosuccinates, alkylamide sulfosuccinates, alkyl sulfoacetates, acylsarcosinates, acylglutamates, alkylsulfosuccinamates, acylisethionates and N-acyltaurates, salts of alkyl monoesters and polyglycoside-polycarboxylic acids, acyllactylates, salts of D-galactoside uronic acids, salts of alkyl ether carboxylic acids, salts of alkyl aryl ether carboxylic acids, and salts of alkylamido ether carboxylic acids; or the non-salified forms of all of these compounds, the alkyl and acyl groups of all of these compounds containing from 6 to 24 carbon atoms and the aryl group denoting a phenyl group. Some of these compounds may be oxyethylenated and then preferably comprise from 1 to 50 ethylene oxide units.

Useful alkyl sulfonates include those of formula (III):

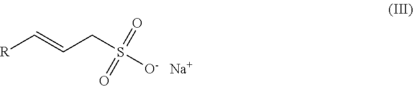

wherein R is selected from H or alkyl chain that has from 1 to 30 carbon atoms, such as from 6 to 24 carbon atoms, for example from 8 to 20 carbon atoms, said chain being saturated or unsaturated, linear or branched. It should be noted that although sodium is shown as the cation in the above formula (III), the cation may be any alkali metal ion such as sodium or potassium, ammonium ions, or alkanolammonium ions such as monoethanolammonium or triethanolammonium ions. In some instances, the alkyl sulfonate(s) are selected from C8-C16 alkyl benzene sulfonates, C10-C20 paraffin sulfonates, C10-C24 olefin sulfonates, salts thereof, and mixtures thereof.

By way of non-limiting example, alkyl sulfonates may be chosen from alkyl aryl sulfonates, primary alkane disulfonates, alkene sulfonates, hydroxyalkane sulfonates, alkyl glyceryl ether sulfonates, alpha-olefinsulfonates, sulfonates of alkylphenolpolyglycol ethers, alkylbenzenesulfonates, phenvlalkanesulfonates, alpha-olefinsulfonates, olefin sulfonates, alkene sulfonates, hydroxyalkanesulfonates and disulfonates, secondary alkanesulfonates, paraffin sulfonates, ester sulfonates, sulfonated fatty acid glycerol esters, and alpha-sulfo fatty acid methyl esters including methyl ester sulfonate.

Non-limiting examples of useful alkyl sulfosuccinates include those of formula (IV):

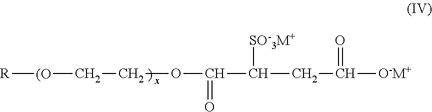

wherein R is a straight or branched chain alkyl or alkenyl group having from 10 to 22 carbon atoms, such as 10 to 20 carbon atoms, and M⁺ is a cation which can independently of each other be, for example, any alkali metal ion such as sodium, potassium, or ammonium, or alkanolammonium ions such as monoethanolammonium or triethanolammonium ions.

Non-limiting examples of alkyl sulfosuccinates salts include disodium oleamido MIPA sulfosuccinate, disodium oleamido MEA sulfosuccinate, disodium lauryl sulfosuccinate, disodium laureth sulfosuccinate, diammonium lauryl sulfosuccinate, diammonium laureth sulfosuccinate, dioctyl sodium sulfosuccinate, disodium oleamide MEA sulfosuccinate, sodium dialkyl sulfosuccinate, and a mixture thereof.

Exemplary and non-limiting alkyl sulfoacetates include C4-C18 fatty alcohol sulfoacetates and/or salts thereof, such as sodium lauryl sulfoacetate. Useful cations for the salts include any alkali metal ion such as sodium or potassium, ammonium ions, or alkanolammonium ions such as monoethanolammonium or triethanolammonium ions.

Non-limiting examples of alkoxylated monoacids include compounds corresponding to formula (V):

R—O[CH₂O]ᵤ[(CH₂)ₓCH(R′)(CH₂)_{y}(CH₂)_{z}O]ᵥ[CH₂CH₂O]_{w}CH₂—COOH (V)

wherein:
R is a hydrocarbon radical containing from about 6 to about 40 carbon atoms;
u, v, and w, independently of one another, represent numbers of from 0 to 60;
x, y, and z, independently of one another, represent numbers of from 0 to 13;
R′ represents hydrogen or alkyl; and
the sum of x+y+z>0.

Compounds corresponding to formula (V) may be obtained by alkoxylation of alcohols R—OH with ethylene oxide as the sole alkoxide, or with several alkoxides and subsequent oxidation. The numbers u, v, and w each represent the degree of alkoxylation. Whereas, on a molecular level, the numbers u, v, and w and the total degree of alkoxylation can only be integers, including zero, on a macroscopic level they are mean values in the form of broken numbers.

In formula (V), R is linear or branched, acyclic or cyclic, saturated or unsaturated, aliphatic or aromatic, substituted or unsubstituted. R may be a linear or branched, acyclic C6-40 alkyl or alkenyl group, or a C1-40 alkyl phenyl group, for example a C8-22 alkyl or alkenyl group or a C4-18 alkyl phenyl group, such as a C12-18 alkyl group or alkenyl group or a C6-16 alkyl phenyl group; u, v, w, independently of one another, may be a number from 2 to 20, for example a number from 3 to 17, such as a number from 5 to 15; x, y, z, independently of one another, may be a number from 2 to 13, for example a number from 1 to 10, such as a number from 0 to 8.

By way of example only, useful alkoxylated monoacids include Butoxynol-5 Carboxylic Acid, Butoxynol-19 Carboxylic Acid, Capryleth-4 Carboxylic Acid, Capryleth-6 Carboxylic Acid, Capryleth-9 Carboxylic Acid, Ceteareth-25 Carboxylic Acid, Coceth-7 Carboxylic Acid, C9-11 Pareth-6 Carboxylic Acid, C11-15 Pareth-7 Carboxylic Acid, C12-13 Pareth-5 Carboxylic Acid, C12-13 Pareth-8 Carboxylic Acid, C12-13 Pareth-12 Carboxylic Acid, C12-15 Pareth-7 Carboxylic Acid, C12-15 Pareth-8 Carboxylic Acid, C14-15 Pareth-8 Carboxylic Acid, Deceth-7 Carboxylic Acid, Laureth-3 Carboxylic Acid, Laureth-4 Carboxylic Acid, Laureth-5 Carboxylic Acid, Laureth-6 Carboxylic Acid, Laureth-8 Carboxylic Acid, Laureth-10 Carboxylic Acid, Laureth-11 Carboxylic Acid, Laureth-12 Carboxylic Acid, Laureth-13 Carboxylic Acid, Laureth-14 Carboxylic Acid, Laureth-17 Carboxylic Acid, PPG-6-Laureth-6 Carboxylic Acid, PPG-8-Steareth-7 Carboxylic Acid, Myreth-3 Carboxylic Acid, Myreth-5 Carboxylic Acid, Nonoxynol-5 Carboxylic Acid, Nonoxynol-8 Carboxylic Acid, Nonoxynol-10 Carboxylic Acid, Octeth-3 Carboxylic Acid, Octoxynol-20 Carboxylic Acid, Oleth-3 Carboxylic Acid, Oleth-6 Carboxylic Acid, Oleth-10 Carboxylic Acid, PPG-3-Deceth-2 Carboxylic Acid, Capryleth-2 Carboxylic Acid, Ceteth-13 Carboxylic Acid, Deceth-2 Carboxylic Acid, Hexeth-4 Carboxylic Acid, Isosteareth-6 Carboxylic Acid, Isosteareth-11 Carboxylic Acid, Trudeceth-3 Carboxylic Acid, Trideceth-6 Carboxylic Acid, Trideceth-8 Carboxylic Acid, Trideceth-12 Carboxylic Acid, Trideceth-3 Carboxylic Acid, Trideceth-4 Carboxylic Acid, Trideceth-7 Carboxylic Acid, Trideceth-15 Carboxylic Acid, Trideceth-19 Carboxylic Acid, Undeceth-5 Carboxylic Acid and mixtures thereof. In some cases, preferred ethoxylated acids include Oleth-10 Carboxylic Acid, Laureth-5 Carboxylic Acid, Laureth-11 Carboxylic Acid, and mixtures thereof.

Acyl amino acids that may be used include, but are not limited to, amino acid surfactants based on alanine, arginine, aspartic acid, glutamic acid, glycine, isoleucine, leucine, lysine, phenylalanine, serine, tyrosine, valine, sarcosine, threonine, and taurine. The most common cation associated with the acyl amino acid can be sodium or potassium. Alternatively, the cation can be an organic salt such as triethanolamine (TEA) or a metal salt. Non-limiting examples of useful acyl amino acids include those of formula (VI):

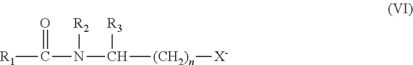

wherein R, R1, R2, and R3 are each independently selected from H or an alkyl chain having from 1 to 30 carbon atoms, said chain being saturated or unsaturated, linear or branched, and X is COO⁻ or SO₃⁻.

By way of example, useful acyl amino acids include acyl taurates, acyl glycinates, acyl glutamates, acyl sarcosinates, salts thereof, and mixtures thereof.

Exemplary useful acyl taurates include those of formula (VII):

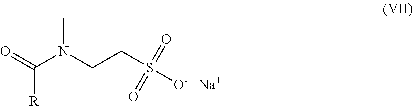

wherein R is selected from H or an alkyl chain having from 1 to 30 carbon atoms, such as from 6 to 24 carbon atoms, for example from 8 to 20 carbon atoms, said chain being saturated or unsaturated, linear or branched. It should be noted that although sodium is shown as the cation in the above formula (VII), the cation may be any alkali metal ion such as sodium or potassium, ammonium ions, or alkanolammonium ions such as monoethanolammonium or triethanolammonium ions. Non-limiting examples of acyl taurate salts include sodium cocoyl taurate, sodium methyl cocoyl taurate, and mixtures thereof.

Exemplary useful acyl glycinates include those of formula (VIII):

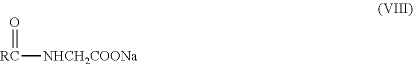

wherein R is an alkyl chain of 8 to 16 carbon atoms. It should be noted that although sodium is shown as the cation in the above formula (VIII), the cation may be any alkali metal ion such as sodium, potassium, or ammonium ions, or alkanolammonium ions such as monoethanolammonium or triethanolammonium ions. Non-limiting examples of acyl glycinates include sodium cocoyl glycinate, sodium lauroyl glycinate, sodium myristoyl glycinate, potassium lauroyl glycinate, and potassium cocoyl glycinate, and mixtures thereof.

Exemplary useful acyl glutamates include those of formula (IX):

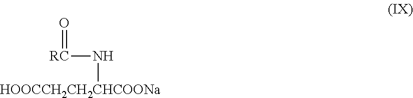

wherein R is an alkyl chain of 8 to 16 carbon atoms. It should be noted that although sodium is shown as the cation in the above formula (IX), the cation may be any alkali metal ion such as sodium, potassium, or ammonium ions, or alkanolammonium ions such as monoethanolammonium or triethanolammonium ions. Non-limiting examples of acyl glutamates include dipotassium capryloyl glutamate, dipotassium undecylenoyl glutamate, disodium capryloyl glutamate, disodium cocoyl glutamate, disodium lauroyl glutamate, disodium stearoyl glutamate, disodium undecylenoyl glutamate, potassium capryloyl glutamate, potassium cocoyl glutamate, potassium lauroyl glutamate, potassium myristoyl glutamate, potassium stearoyl glutamate, potassium undecylenoyl glutamate, sodium capryloyl glutamate, sodium cocoyl glutamate, sodium lauroyl glutamate, sodium myristoyl glutamate, sodium olivoyl glutamate, sodium palmitoyl glutamate, sodium stearoyl glutamate, sodium undecylenoyl glutamate, triethanolamine mono-cocoyl glutamate, triethanolamine lauroylglutamate, disodium cocoyl glutamate, and mixtures thereof.

Non-limiting examples of acyl sarcosinates include potassium lauroyl sarcosinate, potassium cocoyl sarcosinate, sodium cocoyl sarcosinate, sodium lauroyl sarcosinate, sodium myristoyl sarcosinate, sodium oleoyl sarcosinate, sodium palmitoyl sarcosinate, ammonium lauroyl sarcosinate, and mixtures thereof.

When the anionic surfactant(s) are in salt form, they may be chosen especially from alkali metal salts such as the sodium or potassium salt and preferably the sodium salt, ammonium salts, amine salts and in particular amino alcohol salts, or alkaline-earth metal salts such as the magnesium salt. Examples of amino alcohol salts that may especially be mentioned include monoethanolamine, diethanolamine and triethanolamine salts, monoisopropanolamine, diisopropanolamine or triisopropanol-amine salts, 2-amino-2-methyl-1-propanol salts, 2-amino-2-methyl-1,3-propanediol salts and tris(hydroxymethyl)aminomethane salts. Alkali metal or alkaline-earth metal salts and in particular the sodium or magnesium salts may be used. Exemplary salts of C₆-C₂₄ alkyl monoesters of polyglycoside-polycarboxylic acids include C₆-C₂₄ alkyl polyglycoside-citrates, C₆-C₂₄ alkyl polyglycoside-tartrates and C₆-C₂₄ alkyl polyglycoside-sulfo succinates.

In various embodiments, the internal core formulation comprises at least one cationic surfactant. By way of example, optionally polyoxyalkylenated primary, secondary, or tertiary fatty amine salts, quaternary ammonium salts, and mixtures thereof may be chosen.

For example, quaternary ammonium salts may be chosen from those of formula (X):

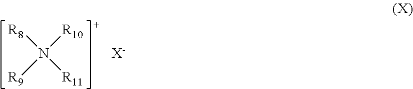

wherein:
R8 to R11 are independently chosen from linear or branched aliphatic groups containing from 1 to 30 carbon atoms, or an aromatic group such as aryl or alkylaryl, at least one of the groups R8 to R11 containing from 8 to 30 carbon atoms and preferably from 12 to 24 carbon atoms; it being possible for the aliphatic groups to comprise heteroatoms such as, in particular, oxygen, nitrogen, sulfur or halogens; and
X⁻ is an anion chosen from the group consisting of halides, phosphates, acetates, lactates, (C1-C4)alkyl sulfates, (C1-C4)alkylsulfonates and (C1-C4)alkylarylsulfonates.

In formula (X), the aliphatic groups may be chosen, for example, from C1-C30 alkyl, C1-C30 alkoxy, (C2-C6)polyoxyalkylene, C1-C30 alkylamide, (C12-C22)alkyl(C2-C6)alkylamido, (C12-C22)alkyl acetate and C1-C30 hydroxyalkyl groups.

As non-limiting examples, tetraalkylammonium chlorides such as, for example, dialkyldimethylammonium or alkyltrimethylammonium chlorides in which the alkyl group comprises approximately from 12 to 22 carbon atoms, particularly behenyltrimethylammonium, distearyldimethylammonium, cetyltrimethylammonium, and benzyldimethylstearylammonium chlorides, palmitylamidopropyltrimethylammonium chloride, or stearamidopropyldimethyl(myristyl acetate) ammonium chloride, which is sold under the name Ceraphyl® 70 by the company Van Dyk may be chosen.

In further exemplary embodiments, quaternary ammonium salts may be chosen from imidazoline quaternary ammonium salts of formula (XI):

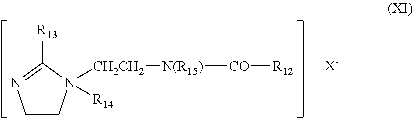

wherein:
R12 represents an alkenyl or alkyl group comprising from 8 to 30 carbon atoms, for example fatty acid derivatives of tallow;
R13 represents a hydrogen atom, a C1-C4 alkyl group or an alkenyl or alkyl group comprising from 8 to 30 carbon atoms;
R14 represents a C1-C4 alkyl group;
R15 represents a hydrogen atom or a C1-C4 alkyl group; and
X⁻ is an anion chosen from the group consisting of halides, phosphates, acetates, lactates, (C1-C4)alkyl sulfates, (C1-C4)alkylsulfonates and (C1-C4)alkylarylsulfonates.

In various embodiments of formula (XI), R12 and R13 preferably denote a mixture of alkenyl or alkyl groups containing from 12 to 21 carbon atoms, for example fatty acid derivatives of tallow, R14 denotes a methyl group, and R15 denotes a hydrogen atom. A product of this kind is sold for example under the name Rewoquat® W 75 by the company Rewo.

In yet further embodiments, quaternary ammonium salts may be chosen from quaternary di- or triammonium salts of formula (XII):

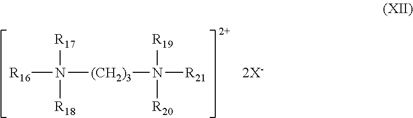

wherein:
R16 denotes an alkyl group containing approximately from 16 to 30 carbon atoms, which is optionally hydroxylated and/or interrupted with one or more oxygen atoms, R17 is chosen from hydrogen or an alkyl group containing from 1 to 4 carbon atoms or a group —(CH2)3-N+(R16a)(R17a)(R18a);
R16a, R17a, R18a, R18, R19, R20 and R21, which may be identical or different, are chosen from hydrogen and an alkyl group comprising from 1 to 4 carbon atoms; and
X⁻ is an anion chosen from the group consisting of halides, acetates, phosphates, nitrates, (C1-C4)alkyl sulfates, (C1-C4)alkylsulfonates and (C1-C4)alkylarylsulfonates, in particular methyl sulfate and ethyl sulfate.

Exemplary compounds include Finquat® CT-P, sold by the company Finetex (Quaternium 89), and Finquat® CT, sold by the company Finetex (Quaternium 75).

In still further embodiments, quaternary ammonium salts may be chosen from quaternary ammonium salts containing one or more ester functions of formula (XIII):

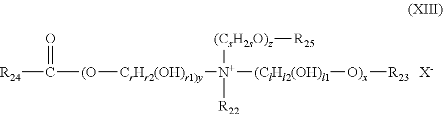

wherein:
R22 is chosen from C1-C6 alkyl groups and C1-C6 hydroxyalkyl or C1-C6 dihydroxyalkyl groups;
R23 is chosen from the group R26-C(═O)—; hydrocarbon-based linear or branched, saturated or unsaturated C1-C22 groups R27; and a hydrogen atom;
R25 is chosen from the group R28-C(═O)—; hydrocarbon-based linear or branched, saturated or unsaturated C1-C6 groups R29; and a hydrogen atom;
R24, R26 and R28, which may be identical or different, are chosen from linear or branched, saturated or unsaturated C7-C21 hydrocarbon-based groups;
r, s and t, which may be identical or different, are integers ranging from 2 to 6;
r1 and t1, which may be identical or different, are equal to 0 or 1;
r2+r1=2r, and t1+t2=2t;
y is an integer ranging from 1 to 10;
x and z, which may be identical or different, are integers ranging from 0 to 10;

and
X⁻ is a simple or complex, organic or mineral anion;

with the provisos that:
the sum x+y+z is from 1 to 15,
when x is 0, then R23 denotes R27, and
when z is 0, then R25 denotes R29.

In formula (XIII), the alkyl groups R22 may optionally be linear or branched, and are preferably linear. Preferably, R22 denotes a methyl, ethyl, hydroxyethyl or dihydroxypropyl group, and more particularly a methyl or ethyl group. Further, the sum x+y+z is preferably from 1 to 10; when R23 is an R27 hydrocarbon group, it may be long and may have from 12 to 22 carbon atoms, or may be short and may have from 1 to 3 carbon atoms; when R25 is an R29 hydrocarbon group, it preferably has 1 to 3 carbon atoms; and when R24, R26 and R28, which are identical or different, are chosen from linear or branched, saturated or unsaturated C11-C21 hydrocarbon groups, and more particularly from linear or branched, saturated or unsaturated C11-C21 alkyl and alkenyl groups; x and z, which may be identical or different, are equal to 0 or 1. Advantageously, y is equal to 1. Preferably, r, s and t, which may be identical or different, are equal to 2 or 3, and even more particularly are equal to 2. Further, X⁻ is preferably a halide, preferably chloride, bromide or iodide, a (C1-C4)alkyl sulfate, (C1-C4)alkyl sulfonate or (C1-C4)alkylaryl sulfonate. However, it is possible to use methanesulfonate, phosphate, nitrate, tosylate, an anion derived from an organic acid, such as acetate or lactate, or any other anion that is compatible with the ammonium comprising an ester function.

Alternatively, R22 denotes a methyl or ethyl group, x and y are equal to 1, z is equal to 0 or 1, r, s and t are equal to 2, R23 is chosen from the group R26-C(═O)—; methyl groups, ethyl groups or hydrocarbon-based C14-C22 groups; and a hydrogen atom, R25 is chosen from the group R28-C(═O)—; and a hydrogen atom, and R24, R26 and R28, which may be identical or different, are chosen from linear or branched, saturated or unsaturated C13-C17 hydrocarbon groups, and preferably from linear or branched, saturated or unsaturated C13-C17 alkyl and alkenyl groups. By way of non-limiting example, compounds that may be mentioned include salts, in particular the chloride or methyl sulfate of diacyloxyethyldimethylammonium, diacyloxyethylhydroxyethylmethylammonium, monoacyloxyethyldihydroxyethylmethyl-ammonium, triacyloxyethylmethylammonium or monoacyloxyethylhydroxyethyldimethyl-ammonium, and mixtures thereof. The acyl groups preferably contain 14 to 18 carbon atoms and are obtained more particularly from a plant oil, such as palm oil or sunflower oil. When the compound contains several acyl groups, these groups may be identical or different.

Preferably, the cationic surfactants are chosen from cetyltrimethylammonium, behenyltrimethylammonium, and dipalmitoylethylhydroxyethyl-methylammonium salts, and mixtures thereof.

In certain embodiments, the internal core formulation comprises at least one non-ionic surfactant. Non-limiting examples of non-ionic surfactants that can be used include fatty alcohols (e.g. cetyl alcohol, cetearyl alcohol), fatty α-diols, fatty (C1-20)alkylphenols, and fatty acids, these compounds optionally being polyethoxylated, polypropoxylated, or polyglycerolated and containing at least one fatty chain comprising, for example, from 8 to 18 carbon atoms, the number of ethylene oxide or propylene oxide groups possibly ranging especially from 2 to 50, and the number of glycerol groups possibly ranging especially from 2 to 30. As exemplary and non-limiting non-ionic surfactants, alkyl polyglucosides and/or monounsaturated glyceryl esters may be chosen.

For example, the internal core formulation may include at least one alkyl polyglucoside having the following formula (XIV):

R¹—O—(R²O)ₙ—Z(x) (XIV)

wherein:
R¹ is an alkyl group having from 8 to 18 carbon atoms;
R² is an ethylene or propylene group;
Z is a saccharide group with 5 to 6 carbon atoms;
n is an integer from 0 to 10; and
x is an integer from 1 to 5.

Nonlimiting examples of alkyl polyglucosides include lauryl glucoside, octyl glucoside, decyl glucoside, coco glucoside, caprylyl/capryl glucoside, and sodium lauryl glucose carboxylate. In certain embodiments, the at least one alkyl polyglucoside compound is selected from the group consisting of lauryl glucoside, decyl glucoside, coco glucoside, and mixtures thereof. Optionally, glucamide surfactants such as alkyl N-methyl glucamide (e.g. cocoyl methyl glucamide) may also be chosen.

In various exemplary embodiments, the non-ionic surfactants chosen from monounsaturated glyceryl esters may include, for example, glyceryl behenate, glyceryl caprate, glyceryl cocoate, glyceryl erucate, glyceryl hydroxystearate, glyceryl isostearate, glyceryl lanolate, glyceryl laurate, glyceryl linoleate, glyceryl myristate, glyceryl oleate, glyceryl palmitate lactate, glyceryl sesquioleate, glyceryl stearate, glyceryl stearate citrate, glyceryl stearate lactate, or mixtures thereof. In certain embodiments, the at least one monounsaturated glyceryl ester may be chosen from polyglyceryl-4 isostearate, polyglyceryl-3 oleate, polyglyceryl-2 sesquioleate, triglyceryl diisostearate, diglyceryl monooleate, tetraglyceryl monooleate, or mixtures thereof.

In yet further exemplary embodiments, the internal core formulation may comprise at least one amphoteric surfactant. Non-limiting examples of amphoteric surfactants include betaines, sultaines, amphoacetates, amphoproprionates, and mixtures thereof. In certain embodiments, betaines and/or amphoproprionates are used. In preferred embodiments, betaines are used. Betaines which can be used in the current compositions include those having the following formulae (XV)-(XVIII):

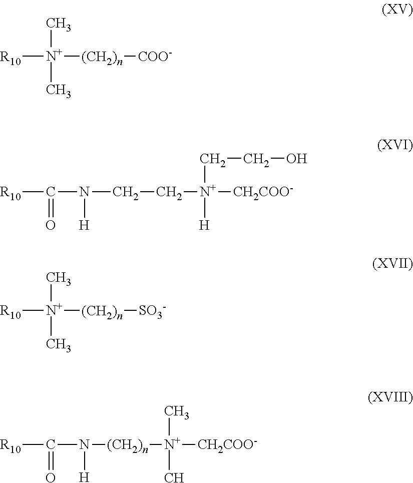

wherein in formulae (XV)-(XVIII):
R10 is an alkyl group having from 8 to 18 carbon atoms; and
n is a integer from 1 to 3.

Non-limiting examples of betaines include coco betaine, cocoamidopropyl betaine, lauryl betaine, laurylhydroxy sulfobetaine, lauryldimethyl betaine, cocoamidopropyl hydroxysultaine, behenyl betaine, capryl/capramidopropyl betaine, lauryl hydroxysultaine, stearyl betaine, and mixtures thereof. In certain embodiments, the at least one betaine compound may be chosen from the group consisting of coco betaine, cocoamidopropyl betaine, behenyl betaine, capryl/capramidopropyl betaine, lauryl betaine, and mixtures thereof.

Hydroxyl sultaines useful in the compositions according to embodiments of the disclosure include the following formula (XIX):

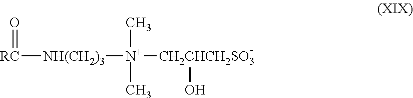

wherein R is an alkyl group having from 8 to 18 carbon atoms.

Useful alkylamphoacetates include those having the formula (XX):

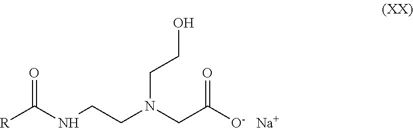

wherein R is an alkyl group having from 8 to 18 carbon atoms.

Useful alkyl amphodiacetates include those having the formula (XXI):

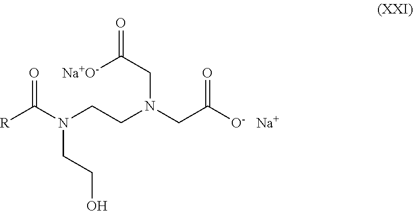

wherein R is an alkyl group having from 8 to 18 carbon atoms.

The amphoteric surfactants of the present disclosure may be optionally quaternized secondary or tertiary aliphatic amine derivatives, in which the aliphatic group is a linear or branched chain comprising from 8 to 22 carbon atoms, said amine derivatives containing at least one anionic group, for instance a carboxylate, sulfonate, sulfate, phosphate or phosphonate group.

Mention may be made in particular of (C₈-C₂₀)alkylbetaines, (C₈-C₂₀)alkylamido (C₁-C₆)alkylbetaines, sulfobetaines, (C₈-C₂₀)alkylsulfobetaines, (C₈-C₂₀)alkylamido(C₁-C₆)alkylsulfobetaines, (C₈-C₂₀)alkylamphoacetate, (C₈-C₂₀)alkyl-amphodiacetate, and mixtures thereof.

Among the optionally quaternized secondary or tertiary aliphatic amine derivatives that may be used, mention may also be made of the products of respective structures (A1) and (A2) below:

Ra—CON(Z)CH₂-(CH₂)ₘ—N⁺(Rb)(Rc)(CH₂COO⁻) (A1)

wherein:
Ra represents a C₁₀-C₃₀ alkyl or alkenyl group derived from an acid Ra-COOH preferably present in hydrolysed coconut oil, a heptyl group, a nonyl group or an undecyl group,
Rb represents a β-hydroxyethyl group,
Rc represents a carboxymethyl group;
m is equal to 0, 1 or 2, and
Z represents a hydrogen atom or a hydroxyethyl or carboxymethyl group;

Ra′-CON(Z)CH₂—(CH₂)_{m′}—N(B)(B′) (A2)

wherein:
B represents —CH₂CH₂OX′, with X′ representing —CH₂-COOH, CH₂-COOZ′, CH₂CH₂-COOH, —CH₂CH₂-COOZ′, or a hydrogen atom,
B′ represents —(CH₂)_{z}-Y′, with z=1 or 2, and Y′ representing COOH, COOZ′, CH₂—CHOH-SO₃H or —CH₂—CHOH—SO₃Z′,
m′ is equal to 0, 1 or 2,
Z represents a hydrogen atom or a hydroxyethyl or carboxymethyl group,
Z′ represents an ion resulting from an alkali or alkaline-earth metal, such as sodium, potassium or magnesium; an ammonium ion; or an ion resulting from an organic amine and in particular from an amino alcohol, such as monoethanolamine, diethanolamine and triethanolamine, monoisopropanol-amine, diisopropanolamine or triisopropanolamine, 2-amino-2-methyl-1-propanol, 2-amino-2-methyl-1,3-propanediol and tris(hydroxymethyl)aminomethane, and
Ra′ represents a C₁₀-C₃₀ alkyl or alkenyl group of an acid Ra′COOH preferably pre-sent in hydrolysed linseed oil or coconut oil, an alkyl group, in particular a C₁₇ alkyl group, and its iso form, or an unsaturated C₁₇ group.

Exemplary amphoteric surfactants include sodium cocoamphoacetate, sodium lauroamphoacetate, sodium caproamphoacetate and sodium capryloamphoacetate. Further exemplary amphoteric surfactants include disodium cocoamphodiacetate, disodium lauroamphodiacetate, disodium caproamphodiacetate, disodium capryloamphodiacetate, disodium cocoamphodi-propionate, disodium lauroam-phodipropionate, disodium caproamphodipropionate, disodium capryloamphodipropionate, lauroamphodipropionic acid and coco-amphodipropionic acid.

Non-limiting examples that may be mentioned include the cocoamphodiacetate sold by the company Rhodia under the trade name Miranol® C2M Concentrate, the sodium cocoamphoacetate sold under the trade name Miranol Ultra C 32 and the product sold by the company Chimex under the trade name CHIMEXANE HA.

Use may also be made of the compounds of formula (XXII):

Ra″-NH—CH(Y″)—(CH₂)ₙ—C(O)—NH—(CH₂)_{n′}—N(Rd)(Re) (XXII)

wherein:
Ra″ represents a C₁₀-C₃₀ alkyl or alkenyl group of an acid Ra″-C(O)OH preferably present in hydrolysed linseed oil or coconut oil;
Y″ represents the group —C(O)OH, —C(O)OZ″, —CH₂—CH(OH)—SO₃H or the group CH₂—CH(OH)—SO₃—Z″, with Z″ representing a cationic counterion resulting from an alkali metal or alkaline-earth metal, such as sodium, an ammonium ion or an ion resulting from an organic amine;
Rd and Re represent, independently of each other, a C₁-C₄ alkyl or hydroxyalkyl radical; and
n and n′ denote, independently of each other, an integer ranging from 1 to 3.

Exemplary compounds include sodium diethylaminopropylcoco-aspartamide, such as the one sold by the company Chimex under the name CHIMEXANE HB.

In certain embodiments, the amphoteric surfactants are chosen from (C₈-C₂₀)alkylbetaines, (C₈-C₂₀)alkylamido(C₁-C₆)alkylbetaines, (C₈-C₂₀)alkylamphoacetates and (C₅-C₂₀)alkylamphodiacetates, and mixtures thereof.

In certain embodiments, the at least one amphoteric surfactant is chosen from (C₈-C₂₀)alkyl betaines, (C₈-C₂₀)alkylamido (C₁-C₆)alkylbetaines, (C₈-C₂₀)alkylamphoacetate, (C₅-C₂₀)alkylamphodiacetate, and their salts, and mixtures thereof. In some cases, the at least one amphoteric surfactant is selected from coco-betaine, cocamidopropylbetaine, sodium cocoamphoacetate, disodium cocoamphodiacetate, and mixtures thereof.

The at least one surfactant may, in various embodiments, be present in the internal core formulations according to the disclosure in an amount ranging up to about 100%, up to about 90%, up to about 80%, up to about 70%, up to about 60%, up to about 50%, up to about 40%, up to about 30%, up to about 20%, up to about 10%, up to about 5%, up to about 3%, or up to about 1%, such as, for example, from about 1% to about 30%, such as about 1% to about 10% or about 1% to about 5%, about 2% to about 25%, such as about 2% to about 10%, about 3% to about 20%, such as about 3% to about 10%, about 4% to about 15%, or about 5% to about 10%, or may be present in an amount ranging from about 30% to about 100%, from about 35% to about 90%, from about 40% to about 80%, or about 50% to about 75% by weight, relative to the total weight of the internal core formulation, including all ranges and sub-ranges thereof.

### Conditioning Agents

The internal core formulations according to the disclosure may optionally comprise one or more conditioning agents, for example either in addition to one or more surfactants (e.g. a dual cleansing/conditioning formulation) or in a formulation having no or minimal surfactants (e.g. a conditioning or moisturizing formulation). Exemplary and non-limiting conditioning agents include plant oils, mineral oils, non-volatile linear silicones such as those of polydialkylsiloxane structure, of polydiarylsiloxane structure, or of polyalkylarylsiloxane structure, fatty esters including fatty acid diesters of polyethylene glycol comprising from 2 to 50 oxyethylene units, cationic polymers, and mixtures thereof.

As used herein, the term “oil” includes any nonionic lipophilic compound that is insoluble in water and liquid at room temperature (25° C.) and atmospheric pressure. For the purposes of the present disclosure, “water-insoluble” refers to a compound whose solubility at spontaneous pH in water at 25° C. and atmospheric pressure is less than 1%, for example less than 0.5%. The oils may have a melting point of less than 5° C. and a viscosity less than 500 cPs at 25° C., at a shear rate of 1 s⁻¹.

In various embodiments, the conditioning agent may be chosen from plant oils. A “plant oil” is a cosmetically acceptable oil as defined above, obtained from a species belonging to the plant kingdom. Exemplary and non-limiting plant oils that may be used include sweet almond oil, apricot kernel oil, argan oil, babassu oil, avocado oil, groundnut oil, candlenut oil, camellia oil, camelina oil, safflower oil, beauty-leaf oil, rapeseed oil, coconut oil, coriander oil, marrow oil, wheatgerm oil, jojoba oil, linseed oil, macadamia oil, corn germ oil, hazelnut oil, walnut oil, vernonia oil, olive oil, evening primrose oil, palm oil, passion flower oil, grapeseed oil, pracaxi oil, rose oil, castor oil, rye oil, sesame oil, rice bran oil, soybean oil, tamanu oil, and sunflower oil.

Essential oils may also be used. Non-limiting examples include essential oil of cinnamon, essential oil of ginger, essential oil of black pepper, essential oil of pimento leaf, essential oil of peppermint and essential oil of clove, and mixtures thereof.

The at least one conditioning agent may be chosen from mineral oils. The term “mineral oils” means hydrocarbons in the form of linear or branched, saturated or unsaturated oils, of mineral or synthetic origin, and which may be hydrogenated. As examples of mineral oils that may be used in the present invention, mention may be made of mixtures of hydrocarbon-based oils derived from petroleum (INCI name: Mineral Oil), volatile or non-volatile liquid paraffin, liquid petroleum jelly, polyolefins and in particular polydecenes, isoparaffins such as isohexadecane, isododecane and hydrogenated polyisobutylenes such as Parleam® oil sold by the company NOF Corporation (INCI name: Hydrogenated polyisobutene).

In various embodiments, the conditioning agents may be chosen from non-volatile linear silicones chosen from polydialkylsiloxanes, polydiarylsiloxanes and polyalkylarylsiloxanes. The non-volatile linear silicones may have a viscosity of greater than or equal to 5 cSt at 25° C. According to one exemplary embodiment, this viscosity ranges from 5 cSt to 1,000,000 cSt, such as from 5 cSt to 100,000 cSt, or from 100 to 10,000 cSt.

Exemplary and non-limiting non-volatile linear silicones include those of formula (XXIII):

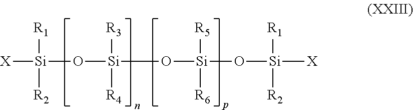

where:
R₁, R₂, R₅ and R₆ are independently chosen from C1-C6 alkyl radicals;
R₃ and R₄ are independently chosen from C1-C6 alkyl radicals or an aryl radical;
X is chosen from C1-C6 alkyl radicals or a hydroxyl radical; and
n and p are integers selected such as to give a compound with a viscosity of more than 5 cSt, where the sum n+p is preferably greater than 10.

In one embodiment, the silicone according to the invention is a non-volatile linear polydimethylsiloxane (PDMS), i.e. where R₁, R₂, R₃, R₄, R₅ and R₆ are identical and denote a methyl radical. In one variation, the polydimethylsiloxane comprises trimethylsilyl end groups, i.e. X denotes a methyl group. In a further variation, the polydimethylsiloxane comprises dimethylsilanol end groups, i.e. X denotes a hydroxyl group.

In various embodiments, the silicones may contain aryl groups, i.e. polydiarylsiloxanes, especially polydiphenylsiloxanes and polyalkylarylsiloxanes.

In further embodiments, the conditioning agents may be chosen from fatty esters. For example, fatty esters having at least 10 carbon atoms may be chosen. These fatty esters include esters derived from fatty acids or alcohols (e.g., mono-esters, polyhydric alcohol esters, and di- and tri-carboxylic acid esters). The fatty esters hereof may include or have covalently bonded thereto other compatible functionalities, such as amides and alkoxy moieties (e.g., ethoxy or ether linkages, etc.).

For example, the fatty esters may be chosen from monoesters comprising at least 18 carbon atoms, such as from 18 and 40 carbon atoms in total, such as monoesters of formula R₁COOR₂, where R₁ represents a linear or branched, saturated or unsaturated or aromatic fatty acid residue comprising from 4 to 40 carbon atoms and R₂ represents a hydrocarbon-based chain that is in particular branched, containing from 4 to 40 carbon atoms, on condition that the sum of the carbon atoms of the radicals R₁ and R₂ is greater than or equal to 18. For instance, Purcellin oil (cetostearyl octanoate), isononyl isononanoate, C₁₂ to C₁₅ alkyl benzoate, 2-ethylhexyl palmitate, octyldodecyl neopentanoate, 2-octyldodecyl stearate, 2-octyldodecyl erucate, isostearyl isostearate, C₁₂-C₁₅ alkyl benzoates such as 2-octyldodecyl benzoate, alcohol or polyalcohol octanoates, decanoates or ricinoleates, isopropyl myristate, isopropyl palmitate, butyl stearate, hexyl laurate, 2-ethylhexyl palmitate, 2-hexyldecyl laurate, 2-octyldecyl palmitate or 2-octyldodecyl myristate, or mixtures thereof may be chosen.

The fatty esters may also be chosen from fatty acid diesters of polyethylene glycol comprising from 2 to 50 oxyethylene units. For example, the fatty acid diesters may be chosen from those of formula (XXIV):

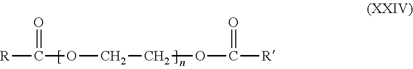

where:
R and R′ are independently chosen from linear or branched alkyl or alkenyl chains comprising from 7 to 29 carbon atoms, preferably from 11 to 23 carbon atoms, more preferably from 15 to 19 carbon atoms, and better still 17 carbon atoms; and
n is an integer ranging from 2 to 50, preferably from 2 to 20 and even more preferentially from 2 to 10.

For example, the fatty acid diester may be a polyethylene glycol distearate comprising from 2 to 50 oxyethylene units, preferably from 2 to 20 oxyethylene units, more preferentially from 2 to 10 oxyethylene units, and better still from 2 to 5 oxyethylene units.

In various embodiments, the conditioning agents may be chosen from cationic polymers. Suitable examples of cationic polymers are those from selected from cationic cellulose derivatives, quaternized hydroxyethyl cellulose, cationic starch derivatives, cationic guar gum derivatives (hydroxypropyl guar hydroxypropyltrimonium chloride, guar hydroxypropyltrimonium chloride), copolymers of acrylamide and dimethyl-diallyammonium chloride, polyquaterniums, and a mixture thereof.

In certain embodiments, the cationic polymer may be selected from cationic guar gum derivatives or guar gums containing cationic trialkylammonium groups, their salts, and a mixture thereof. Non-limiting examples of cationic guar gum derivatives include hydroxypropyl guar hydroxypropyltrimonium chloride, guar hydroxypropyltrimonium chloride, guar gums modified with a 2,3-epoxypropyl-trimethylammonium salt (for example, chloride) and a mixture thereof. Such products are sold especially under the trade names JAGUAR C-13-S guar hydroxypropyltrimonium chloride), JAGUAR C135, JAGUAR C15, JAGUAR C17 and JAGUAR C162 (hydroxypropyl guar hydroxypropyltrimonium chloride) by the company Rhodia (Solvay). Guar hydroxypropyltrimonium chloride may also be sold under the tradename of N-HANCE CG13 by the company Ashland.

Optionally, the cationic polymer may be selected from cationic cellulose derivatives, quaternized hydroxyethyl cellulose, or mixtures thereof. For example, the cationic polymer may be selected from polyquaternium compounds. In various embodiments, the cationic polymer may be selected from hydroxypropyl guar hydroxypropyltrimonium chloride, guar hydroxypropyltrimonium chloride, and a mixture thereof.

The at least one conditioning agent may optionally be present in the internal core formulation in an amount up to about 100%, such as from about 0.1% to about 100% by weight, based on the total weight of the internal core formulation. By way of example, in various embodiments, the at least one conditioning agent may be present in the internal core formulations according to the disclosure in an amount ranging up to about 20%, such as, for example from about 0.1% to about 20%, such as about 1% to about 20%, about 1% to about 15%, about 1% to about 10%, about 1% to about 3%, about 5% to about 20%, about 5% to about 15%, about 5% to about 10%, about 10% to about 20%, or about 10% to about 15% by weight, relative to the total weight of the internal core formulation, including all ranges and sub-ranges thereof. In further embodiments, the at least one conditioning agent may be present in an amount ranging from about 25% to about 100%, such as about 30% to about 95%, about 35% to about 90%, about 40% to about 85%, about 45% to about 80%, about 50% to about 75%, about 55% to about 70%, or about 60% to about 65% by weight, relative to the total weight of the internal core formulation, including all ranges and sub-ranges thereof.

It should be noted that in one embodiment, the internal core formulations are free or substantially free of silicones. For example, the compositions may in some embodiments include less than about 3%, less than about 2%, less than about 1%, or less than about 0.5% of silicones.

### Additional Components

The internal core formulation may optionally comprise a solvent, e.g. water, in an amount up to about 30% by weight, relative to the total weight of the internal core formulation. For example, the internal core formulation may comprise water in an amount ranging from about 1% to about 30%, about 5% to about 25%, or about 10% to about 20% by weight, including all ranges and sub-ranges thereof.

The internal core formulation according to the disclosure may also optionally comprise additives, for example chosen from fatty alcohols, nacreous agents, dyes or pigments, fragrances, mineral, plant or synthetic oils, waxes, vitamins (e.g. panthenol, Vitamin E, biotin, etc.), proteins including ceramides, UV-screening agents, free-radical scavengers, antidandruff agents, hair-loss counteractants, hair restorers, preserving agents, and mixtures thereof. A person skilled in the art will take care to select the optional additives and the amount thereof such that they do not harm the properties of the formulations.

In various embodiments, the additives are generally present in an amount ranging 0.1% to 10% by weight of active material relative to the total weight of the internal core formulation.

While not required, it may be advantageous if the internal core formulation has a melting point of less than about 50° C., such as from about 30° C. to about 50° C., or about 32° C. to about 45° C. This may, for example, improve spreadability of the internal core formula when heated (e.g. by a user's hands or warm water), and/or may facilitate application to the skin, hair, and/or scalp.

### Protective Shell Coating

The internal core of the mono-dose products will be completely, or substantially completely, surrounded by a protective shell coating. Without intending to be limiting, the process and/or shell coating compositions described in CA 2,555,568A1, incorporated by reference in its entirety, may be used. The protective shell coating composition may comprise structuring agents, as well as other components such as gums, film formers, binders, and any other ingredient useful for forming a protective shell around the internal core.

The protective shell coating composition comprises at least one structuring agent. In one embodiment, the at least one structuring agent is chosen from monosaccharides, disaccharides, and polysaccharides, as well as hydrolysis products and derivatives thereof. For example sucrose, fructose, glucose, galactose, lactose, fructans, gellans, glucans, amylose, amylopectin, glycogen, pullulan, dextrans, corn syrup, celluloses and derivatives thereof, in particular methylcelluloses, hydroxyalkylcelluloses, ethylhydroxyethylcelluloses and carboxymethylcelluloses, mannans, xylans, lignins, arabans, galactans, galacturonans, alginate-based compounds, chitin, chitosans, glucuronoxylans, arabinoxylans, xyloglucans, glucomannans, pectic acids and pectins, arabinogalactans, carrageenans, agars, glycosaminoglucans, gum arabics, tragacanth gums, ghatti gums, karaya gums, locust bean gums, galactomannans such as guar gums and nonionic derivatives thereof, in particular hydroxypropyl guar, and ionic derivatives thereof, biopolysaccharide gums of microbial origin, in particular scleroglucan or xanthan gums, mucopolysaccharides, and in particular chondroitin sulfates, starches including hydrolyzed, partially hydrolyzed, native, and modified starches, for example corn starch, rice starch, tapioca starch, cassava starch, barley starch, potato starch, wheat starch, sorghum starch, and pea starch, and sugar alcohols, such as sorbitol, as well as mixtures thereof, may be chosen.

In other embodiments, polymer structuring agents such as carboxylic acid polymers (e.g. carbomer), polyacrylamide polymers, polyacrylates (e.g. copolyacrylamide and C13-14 isoparaffin and laureth-7; polyacrylate crosspolymer-6; acrylamide/ammonium acrylate copolymer (and) polyosobutene (and) polysorbate 20; acrylates/C13-30 alkyl acrylate crosspolymer; sodium polyacryloyldimethyl taurate (and) polysorbate 80 (and) sorbitan anoleate; sodium acrylates copolymer and lecithin) and polyurethanes (e.g. steareth-100/PEG-136/HDI copolymer; BIS-C16-20 Isoalkoxy TMHDI/PEG-90 copolymer) may be chosen. For example, the polymeric structuring agent may be chosen from ammonium polyacryloyldimethyl taurate, ammonium acryloyldimethyltaurate/VP copolymer, sodium polyacrylate, acrylates copolymers, polyacrylamide, carbomer, acrylates/C10-30 alkyl acrylate crosspolymer, or a mixture thereof.

The structuring agent may be present in the protective shell coating composition in an amount ranging up to about 95%, such as about 50% to about 90%, about 60% to about 85%, about 65% to about 80%, about 67% to about 78%, about 68% to about 76%, or about 70% to about 75%, based on the total weight of the protective shell coating composition, including all sub-ranges thereof.

In certain embodiments, the protective shell coating composition comprises at least one gum component. By way of example, the gum may be chosen from gum arabic, xanthan gum, gelatin, gum tragacanth, maltodextrin, and the like, as well as mixtures thereof. The gum component may, for example, be present in an amount ranging up to about 5%, for example ranging from about 2% to about 5%, about 2% to about 4%, or about 3% to about 5% by weight, relative to the total weight of the protective shell coating composition, including all sub-ranges thereof.

In further embodiments, the protective shell coating composition optionally comprises one or more additives, such as at least one film forming agent, at least one binder (e.g. plant gums, carboxymethylcellulose, gelatin or fatty substances, such as monoglycerides, diglycerides, and the like), anti-tack agents, anti-microbial agents, coloring agents (e.g. FD+C Yellow 5 Lake, FD+C Yellow 6 Lake, FD+C Blue 1 Lake, FD+C Blue 2 Lake, FD+C Red #40 Lake, and the like), and/or at least one crystallizing agent.

In various embodiments, the additives may be present in an amount ranging 0.1% to 10% by weight of active material relative to the total weight of the protective shell coating composition.

The protective shell coating composition further comprises a solvent, e.g. water. The solvent may be present in the protective shell coating composition in an amount up to about 40%, such as from about 5% to about 35% by weight, relative to the total weight of the protective shell coating composition. Optionally, the total amount of solids in the protective shell coating composition ranges from about 40% to about 95%, such as about 65% to about 85%, about 68% to about 82%, about 69% to about 80%, or about 70% to about 75% by weight, relative to the total weight of the protective shell coating composition, including all sub-ranges thereof.

The protective shell coating may be applied to the internal core by any method known. By way of example, the protective shell coating may be formed around the internal core by dip coating, spray coating (e.g. using an atomizer), and/or pan/tumble coating. Optionally, the temperature of the internal core formulation may be lowered prior to application of the protective shell coating.

The protective shell coating may be applied to the internal core in one, two, three, or more applications. For example, the protective shell coating may be applied to the internal core formulation and then dried, followed by a second application and second drying, and so on, until the desired coating is formed. In some embodiments where multiple applications of the protective shell coating are employed, it may be desirable to have each application comprise an identical composition as used in previous applications, while in alternate embodiments it may be desirable for one or more applications to comprise a different composition as used in previous applications.

The protective shell coating may have a thickness ranging up to about 3 mm, such as from about 0.01 mm to about 2 mm. While the protective shell coating may be substantially uniform in thickness, it is not required.

In various embodiments, the hardness of the protective shell coating may be greater than the hardness of the internal core formulation. For example, the protective coating may have a hardness of at least 10% greater than the hardness of the internal core formulation, such as at least 15% greater, at least 20% greater, at least 25% greater, at least 30% greater, at least 35% greater, at least 40% greater, at least 45% greater, at least 50% greater, at least 55% greater, at least 60% greater, at least 65% greater, at least 70% greater, at least 75% greater, at least 80% greater, at least 85% greater, at least 90% greater, at least 95% greater, at least 100% greater, at least 105% greater, at least 110% greater, at least 115% greater, at least 120% greater, at least 125% greater, at least 130% greater, at least 135% greater, at least 140% greater, at least 145% greater, at least 150% greater, at least 155% greater, at least 160% greater, at least 165% greater, at least 170% greater, at least 175% greater, at least 180% greater, at least 185% greater, at least 190% greater, at least 195% greater, at least 200% greater, or at least 250% greater than the hardness of the internal core formulation.

### Water-Resistant Coating

Optionally, it may be desirable to have a water-resistant coating covering the protective shell. This may be advantageous, for example, when the mono-dose products and systems are exposed to high humidity, such as in a shower, to prevent degradation or disintegration of the protective shell.

In various embodiments, the water-resistant coating may comprise at least one wax. Although not required, the at least one wax may, in some embodiments, have a melting point of less than about 95° C.

By way of non-limiting example, the at least one wax may be chosen from waxes of animal origin, such as beeswaxes or modified beeswaxes (cera bellina), spermaceti, lanolin wax and lanolin derivatives; plant waxes such as carnauba wax, candelilla wax, esparto wax, ouricury wax, Japan wax, cocoa butter, cork-fibre wax, sugarcane wax, olive-tree wax, rice wax, hydrogenated jojoba wax or absolute waxes of flowers; mineral waxes, for example paraffin wax, petroleum jelly wax, lignite wax, microcrystalline waxes, or ozokerites; synthetic waxes, for example synthetic beeswax or polyethylene wax; and mixtures thereof.

It may, in certain embodiments, be advantageous to use at least two waxes. In various embodiments, the waxes may be chosen from at least two waxes where the waxes have different hardness and/or melting points. For example, it may be beneficial to use a combination of beeswax (softer wax, melting point 52° C.) and carnauba wax (harder wax, melting point 82.3° C.). Useful exemplary waxes and their melting points can be found, for example, in U.S. Pat. No. 9,408,785, incorporated by reference herein.

Optionally, when at least two waxes are chosen, it may be advantageous to have a greater amount of harder wax. By way of illustration only, when beeswax and carnauba wax are chosen, the ratio of beeswax:carnauba wax may range from about 1:10 to about 1:1.25, such as about 1:10, 1:9, 1:8, 1:7, 1:6, 1:5, 1:4, 1:3, 1:2, 1:5, or 1:1.25. It should be understood that these ratios are disclosed for other combinations of harder wax:softer wax as well, including combinations having more than one harder wax and/or more than one softer wax.

In other embodiments when at least two waxes are chosen, it may be advantageous to have a greater amount of softer wax. By way of illustration only, when beeswax and carnauba wax are chosen, the ratio of carnauba wax:beeswax may range from about 1:10 to about 1:1.25, such as about 1:10, 1:9, 1:8, 1:7, 1:6, 1:5, 1:4, 1:3, 1:2, 1:5, or 1:1.25. It should be understood that these ratios are disclosed for other combinations of softer wax:harder wax as well, including combinations having more than one harder wax and/or more than one softer wax.

### Methods of Use

According to various embodiments, the mono-dose systems may be useful in methods of caring for, cleansing, and/or conditioning the skin, hair, and/or scalp. By way of example, when the internal core formulations are shampoo compositions or conditioning shampoo compositions, the mono-dose systems may comprise using one or more mono-dose products in methods of cleansing and/or conditioning the hair and/or scalp. When the internal core formulations are skin care or skin cleansing compositions, the mono-dose systems may comprise using one or more mono-dose products in methods of cleansing and/or caring for the skin. When the internal core formulations are skin care or conditioning compositions, or hair care or conditioning compositions, the mono-dose systems may comprise using one or more mono-dose products in methods of caring for and/or conditioning the skin, hair, and/or scalp.

For example, the mono-dose products may be activated with one or more of heat and/or water and/or pressure (e.g. exposed to sufficient amounts of heat and/or water and/or pressure), in order to release the internal core formulation. For example, one or more mono-dose products may be exposed to heat and/or water, the mono-dose product(s) broken by force such as crushing, breaking, or mechanically rubbing the mono-dose products between the palms of the hands, and optionally a lather or foam may be formed. The internal core formulation or lather or foam then may be applied to the keratinous substrate, e.g. the skin, hair, and/or scalp in a traditional manner for caring for, cleansing, and/or conditioning the keratinous substrate, and optionally rinsed, for example after a leave-in period of time.

It should be noted that, in certain embodiments, heat of the user's hands may be sufficient for activating the mono-dose products according to the disclosure, but additional heat and/or water (e.g. hot water) and/or pressure may in some embodiments accelerate the activation and increase the ease of use of the mono-dose products and systems.

It will be apparent that in some embodiments, more than one mono-dose product may be useful in the methods for caring for, cleansing, and/or conditioning the skin, hair, and/or scalp according to the disclosure. By way of illustrative example only, in an embodiment where the mono-dose system is a system for cleansing the hair, a user with long, thick hair may desire more than one mono-dose product in order to deliver sufficient amounts of shampoo product to the entire head of hair.

It should be understood that various features and/or characteristics of differing embodiments herein may be combined with one another. It is therefore to be understood that numerous modifications may be made to the illustrative embodiments and that other arrangements may be devised without departing from the scope of the disclosure. Other embodiments will be apparent to those skilled in the art from consideration of the disclosure and practice of the various exemplary embodiments disclosed herein.

As used herein, the term “keratinous substrate” is intended to include skin (including the scalp) and hair.

As used herein, the term “mono-dose product” is meant to describe a single-use product (e.g. in the form of a capsule, bead, ball, or the like) for caring for, cleansing, and/or conditioning the skin, hair, and/or scalp.

As used herein, the term “mono-dose system” is meant to describe a system of one or more, such as two or more, three or more, and so on, mono-dose products that is/are used for caring for, cleansing, and/or conditioning the skin, hair, and/or scalp.

As used herein, the terms “comprising,” “having,” and “including” are used in their open, non-limiting sense.

The terms “a,” “an,” and “the” are understood to encompass the plural as well as the singular. Thus, the term “a surfactant” includes both a single surfactant and a plurality of surfactants.

The expression “one or more” means “at least one” and thus includes individual components as well as mixtures/combinations.

Other than in the operating examples, or where otherwise indicated, all numbers expressing quantities of ingredients and/or reaction conditions are to be understood as being modified in all instances by the term “about,” meaning within +/−5%, 4%, 3%, 2%, or 1% of the indicated number. Ranges can be expressed herein as from “about” one particular value, and/or to “about” another particular value. When such a range is expressed, examples include from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

All percentages, parts and ratios herein are relative to the amount of active agent, based upon the total weight of the compositions of the present disclosure, unless otherwise indicated.

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc.

As used herein, the terms “free” and “substantially free” are intended to denote that the component is absent entirely from the composition, or is present in an amount considered by those skilled in the art to not provide an effect on the composition. For example, the component may be present in an amount below the level of detection, or may be present in an amount less than 3%, less than 2%, less than 1%, less than 0.5%, less than 0.1%, less than 0.01%, or less than 0.001%.

It should also be understood that the precise numerical values used in the specification and claims form additional embodiments of the disclosure, and are intended to include any ranges which may be narrowed to any two end points disclosed within the exemplary ranges and values provided, as well as the specific end points themselves. Efforts have been made to ensure the accuracy of the numerical values disclosed herein. Any measured numerical value, however, can inherently contain certain errors resulting from the standard deviation found in its respective measuring technique.

The compositions and methods of the present disclosure can comprise, consist of, or consist essentially of the essential elements and limitations of the disclosure described herein, as well as any additional or optional ingredients, components, or limitations described herein or otherwise useful.

Unless otherwise expressly stated, it is in no way intended that any method set forth herein be construed as requiring that its steps be performed in a specific order. Accordingly, where a method claim does not expressly recite that a particular order of steps must be followed or it is not otherwise specifically stated in the claims or descriptions that the steps are to be limited to a specific order, it is no way intended that any particular order be inferred.

### EXAMPLES

The following examples serve to illustrate the embodiments of the disclosure without, however, exhibiting a limiting character. The Examples are intended to be non-restrictive and explanatory only, with the scope of the invention being defined by the claims. In these examples the amounts of the composition ingredients are given as weight percentages of active ingredients relative to the total weight of the composition.

### Example 1—Mono-Dose Products

The following two-part or three-part mono-dose products can be prepared.

An internal core conditioning/moisturizing formulation can be prepared by traditional methods:

| Classification | INCI US | Wt % |
|---|---|---|
| Non-ionic | Glucamide surfactant and/or fatty | 10%-50% |
| surfactant(s) | alcohols (e.g. cetyl alcohol, cetearyl | |
| | alcohol) | |
| Cationic | Quaternary ammonium compounds | 0-10% |
| surfactant(s) | (e.g, alkyltrimonium chloride | |
| | compounds) | |
| Conditioning | Plant oil (e.g. coconut oil) | 35%-85% |
| Agent(s) | | |
| Thickening | Gum derivatives, cellulose derivatives | 0%-20% |
| Agent(s) | (e.g., guar gum, hydroxypropyl guar, | |
| | hydroxyethylcellulose, celulose gum) | |
| Additional | Coloring agents, vitamins, fatty | 0.1%-10% |
| component(s) | alcohols, preservatives, etc. | |
| Solvent | Water | QS 100 |

An internal core shampoo formulation can be prepared by traditional methods:

| Classification | INCI US | Wt % |
|---|---|---|
| Anionic | Sulfate-based or non-sulfate-based | 50%-75% |
| Surfactant(s) | anionic surfactant (e.g. sodium laureth | |
| | sulfate, sodium lauryl sulfate, | |
| | isethionate surfactant, glutamate | |
| | surfactants, sulfosuccinate surfactants, | |
| | acyl taurates, acyl glycinates, acyl | |
| | glutamates, acyl sarcosinates or salts | |
| | or derivatives) | |
| Conditioning | Plant oil (e.g. coconut oil), cationic | 0%-20% |
| Agent(s) | polymers (e.g., cationic guar | |
| | derivatives) | |
| Thickening | Gum derivatives, cellulose derivatives | 0.1%-20% |
| Agent(s) | (e.g., guar gum, hydroxypropyl guar, | |
| | hydroxyethylcellulose, celulose gum) | |
| Solvent | Water | QS 100 |
| Additional | Coloring agents, vitamins, fatty | 0.1%-10% |
| component(s) | alcohols, preservatives, etc. | |

Optionally, the temperature of the internal core formulations may be lowered, and aliquots of the internal core formulations may be coated in one or more applications with the following protective shell coating composition by dip coating, spray coating, and/or pan/tumble coating:

| Classification | INCI US | Wt % |
|---|---|---|
| Structuring | Glucose and corn syrup | 50%-95% |
| agent(s) | | |
| Solvent | Water | 5%-40% |
| Additional | Film former, binder, anti-tack agent, anti- | 0.1%-10% |
| component(s) | microbial agent, coloring agent, etc. | |

Once the mono-dose products comprising the internal core formulations coated with the protective shell coating are prepared, they may optionally be coated with a water-resistant coating comprising a combination of beeswax and carnauba wax, optionally in a ratio of beeswax:carnauba wax ranging from about 1:10 to about 1:1.25.

### Example 2—Cleansing or Conditioning the Hair or Skin with Mono-Dose Systems

One or more, for example two, of the mono-dose products prepared in Example 1 can be activated using heat and/or water and/or pressure, e.g. exposing the mono-dose products to hot water while mechanically crushing the mono-dose products to release the internal core formulation. The internal core formulation may be applied to the hair to cleanse or condition the hair, followed by rinsing.

1. A mono-dose product for caring for, cleansing, and/or conditioning keratinous substrates, comprising:
(a) an internal core formulation comprising at least one component chosen from:
i) at least one surfactant, and
ii) at least one conditioning agent,
(b) a protective shell coating comprising at least one structuring agent, and
(c) optionally, a water-resistant coating comprising at least one wax.

2. The mono-dose product of claim 1, wherein the internal core formulation comprises at least one surfactant chosen from anionic surfactants, non-ionic surfactants, amphoteric surfactants, cationic surfactants, or mixtures thereof.

3. The mono-dose product of claim 2, wherein the at least one surfactant is present in an amount ranging from about 30% to about 100%, such as about 50% to about 75% by weight, relative to the total weight of the internal core formulation.

4. The mono-dose product of claim 1, wherein the internal core formulation comprises at least one conditioning agent chosen from plant oils, mineral oils, non-volatile linear silicones, fatty acid diesters of polyethylene glycol comprising from 2 to 50 oxyethylene units, fatty esters, cationic polymers, or a mixture thereof.

5. The mono-dose product of claim 4, wherein the at least one conditioning agent is present in an amount ranging from about 0.1% to about 100%, such as about 0.1% to about 20%, about 30% to about 100%, or about 50% to about 90% by weight, relative to the total weight of the internal core formulation.

6. The mono-dose product of claim 1, wherein the internal core formulation further comprises water.

7. The mono-dose product of claim 6, wherein the internal core formulation further comprises at least one additive chosen from nacreous agents, dyes, pigments, fragrances, mineral, plant or synthetic oils, waxes, vitamins, proteins UV-screening agents, free-radical scavengers, antidandruff agents, hair-loss counteractants, hair restorers, preserving agents, or a mixture thereof.

8. The mono-dose product of claim 1, wherein the at least one structuring agent in the protective shell coating is chosen from monosaccharides, disaccharides, polysaccharides, and hydrolysis products and derivatives thereof, and polymeric structuring agents, or a mixture thereof.

9. The mono-dose product of claim 8, wherein the at least one structuring agent is present in the protective shell coating in an amount ranging from about 60% to about 85% by weight, relative to the total weight of the protective shell coating.

10. The mono-dose product of claim 9, wherein the protective shell coating further comprises water.

11. The mono-dose product of claim 8, wherein the protective shell coating comprises at least one gum.

12. The mono-dose product of claim 10, wherein the protective shell coating further comprises at least one component chosen from film forming agents, binders, anti-tack agents, anti-microbial agents, coloring agents, crystallizing agents, or a mixture thereof.

13. The mono-dose product of claim 1, comprising a water-resistant coating, wherein the at least one wax is chosen from waxes of animal origin, waxes of plant origin, mineral waxes, synthetic waxes, or a mixture thereof.

14. The mono-dose product of claim 13, wherein the at least one wax is chosen from beeswax, carnauba wax, or a mixture thereof.

15. The mono-dose product of claim 14, wherein the water-resistant coating comprises a mixture of carnauba wax and beeswax.

16. The mono-dose product of claim 15, wherein the mixture is present in the water-resistant coating in a ratio of beeswax:carnauba ranging from about 1:10 to about 1:1.25.

17. A mono-dose system for caring for, cleansing, and/or conditioning keratinous substrates, comprising at least one mono-dose product comprising:
(a) an internal core formulation comprising at least one component chosen from:
i) at least one surfactant, and/or
ii) at least one conditioning agent,
(b) a protective shell coating comprising at least one structuring agent, and
(c) optionally, a water-resistant coating comprising at least one wax.

18. (canceled)

19. A method of caring for, cleansing, and/or conditioning keratin substrates, the method comprising:
i. activating a mono-dose product with heat and/or water and/or pressure, wherein the mono-dose product comprises:
(a) an internal core formulation comprising at least one component chosen from:
i) at least one surfactant, and
ii) at least one conditioning agent,
(b) a protective shell coating comprising at least one structuring agent, and
(c) optionally, a water-resistant coating comprising at least one wax, to release the internal core formulation, and
ii. applying the internal core formulation to the keratinous substrate.

20. The method according to claim 19, further comprising rinsing the internal core formulation from the keratinous substrate.
